(19) United States
(12) Patent Application Publication
(10) Pub. No.: US 2007/0148693 A1
(43) Pub. Date: Jun. 28, 2007

(54) Circular recombinant plasmid dna constructs and their protein products, methods of preparation and immobilisation of proteins on support

(75) Inventors: Erinc Sahin, Tuzla-Istanbul (TR); Alpay Taralp, Tuzla-Istanbul (TR); Zehra Sayers, Tuzla-Istanbul (TR)

Correspondence Address: LADAS & PARRY, 26 WEST 61ST STREET, NEW YORK, NY, 10023, US

(73) Assignee: SABANCI UNIVERSITESI, Tulza-Istanbul (TR)

(21) Appl. No.: 10/550,226

(22) PCT Filed: Mar. 20, 2003
(86) PCT No.: PCT/TR03/00019

§ 371 Date: Nov. 22, 2006

(51) Int. Cl. G01N 33/53; C12M 1/34

(52) U.S. Cl. 435/7.1; 427/2.11; 435/287.2

(57) ABSTRACT

The invention relates to new circular recombinant plasmid DNA constructs and their protein products, to the use of said protein products in immobilisation, visualisation and quantification of enzymes and proteins on compatible support material. The invention also relates to a method of preparation and immobilisation of the protein on a compatible support material, to the immobilised proteins obtained by said method as well as the use of said immobilised proteins in several applications.

The invention relates to new circular recombinant plasmid DNA constructs and their protein products, to the use of said protein products in immobilization, visualisation and quantification of enzymes and proteins on compatible support material. The invention also relates to a method of preparation and immobilisation of the protein on a compatible support material, to the immobilised proteins obtained by said method as well as the use of said immobilised proteins in several applications.

The invention concerns the general fields of recombinant plasmid DNA construction, recombinant protein expression, protein detection and quantification, protein immobilisation, arraying, orientation and applications of immobilised proteins. The general application of immobilised proteins and enzymes has played a central role in the expansion of biotechnology and synthesis-related industries.

For immobilising the proteins the following technologies are currently being used:
Adsorption technology has the advantage that the protein-surface interaction is non-covalent and thus precludes any potentially disruptive chemical modifications associated with covalent immobilization, affording enzymes that often retain good, albeit perhaps altered biological activity, but with the common disadvantage of leaching of adsorbed enzymes from the surface and being lost in time. Although adsorption has the said disadvantage, other major advantages include rapid and facile preparation of the material.
Encapsulation technology, like the above said adsorption technology, features many advantages associated with non-covalent protein-surface interactions, particularly the very native-like properties of matrix-enclosed enzymes. In addition, enzymes are easily encapsulated and well retained by the support. A related disadvantage however may be poor substrate accessibility, leading to slowed kinetics or lost apparent activity in the case of large substrates.
Cross-linking technology features the advantages and disadvantages normally associated with covalent modification, one advantage being that protein is well retained on the support. A typical disadvantage of covalent modification is the possibility of loss of biological function as a result of chemical reaction or conditions imposed during the immobilization protocol. A potential advantage/disadvantage defined by context is the alteration of biological function. In the case of cross-linking technology, another disadvantage is the waste of useful biocatalyst since clumps of enzymes are often bound together, intertwined with the support. Under such conditions, diffusional limitations may define the effective enzyme concentration to only those residing near the surface. Furthermore, the dynamic mobility of one enzyme may be impeded by virtue of being anchored to a neighbouring enzyme.
Covalent bonding, like above mentioned cross-linking technology, features the advantage of binding enzyme irreversibly to a surface. The observation of poor or altered activity has introduced modifications to the original method, in which a linker strand separating enzyme and surface imposes a distance constraint, thus restoring much native-like activity with possible exception to site-specific alterations normally associated with covalent modification of protein groups. More often than non-covalent methods, the conditions required to achieve covalent bonding may require harsh conditions that lead to enzyme instability and deactivation.

Applicants refer to Methods in Enzymology: Immobilised Enzymes and Cells, Part B, Vol. 135 (K. Mosbach, Ed) Academic Press, 1987, NY, Methods in Enzymology: Immobilised Enzymes and Cells, Part B, Vol.137 (K. Mosbach, Ed) Academic Press, 1988, NY; Immobilisation of enzymes and cells (G.F. Bickerstaff, Ed.) Humana Press, 1997, NJ, USA, Immobilised biomolecules in analysis: A practical approach (T. Cass and F.S. Ligler, Eds) Oxford University Press, 1998, NY. G.P. Royer (1974) Supports for immobilized enzymes CHEMTECH 4, 699.

Until now tags have been largely limited to poly-His for the reason that it binds to a surface through a coordinating Ni ion. An exception is the work of Nock et al. (S. Nock, J. A. Spudich and P. Wagner (1997) “Reversible, site-specific immobilisation of polyarginine-tagged fusion proteins on mica surfaces” FEBS Letters 141, 233-238) Nonspecific polymer-surface interactions have been discussed very nicely in B. Jonsson, B. Lindman, K. Holmberg and B. Kronberg, Interaction of Polymers with Surfaces: in surfactant and polymers in aqueous solution, John Wiley and Sons, 1998, NY.

Alignment of proteins on a surface using related methods are also given in Y. Harada, K. Yasuda, S. Nomra, N. Kajimura and Y. C. Sasaki (1998) Vectorially Oriented Fixation of Membrane-Embedded Bacteriorhodopsin onto an Inert Base” Langmuir 14, 1829-1835.

The following technologies are currently applied in biotechnological fields:

Multiple histidine residues (His-tag) in series have been applied for the selective purification of recombinant proteins. This technology is already being used for chromotagraphic purification of genetically engineered proteins coupled with nickel bearing columns. Applicants refer to Inouye, S. et al. “Aequorea green fluorescent protein: Expression of the gene and fluorescence characteristics of the recombinant protein” FEBS Letters 341 (1994) pages 227-280.

With explosive developments of the proteomic and genomic era, recombinant proteins are sought in large amounts for research, diagnostic and industrial purposes. While large amounts of various proteins can be produced, much research or applied work has experienced setbacks on the basis of poor purity of proteinaceous starting material. To purify proteins further, a great number of techniques and strategies are being used and developed by scientists. One purification strategy in particular uses the affinity of homo-oligomers or special amino acid sequences to appropriate materials such as metal ion bearing surfaces. In this strategy, the cell lysate is applied onto an affinity material for binding. The non-specific proteins of the organism (which do not have the homo-oligomer tail) are washed away. The recombinant protein, which is of interest for production in higher amounts, is retained by this process and a purification is achieved. Applicants refer to Dyke, Michael W. Van; Sirito, Maro and Sawadogo, Michele “Single-step purification of bacterially expressed polypeptides containing an oligo-histidine domain”, Gene, Col.111, Issue 1, 1 Feb. 1992, pages 99-104.

Fluorescent proteins are readily being used for identifying the location of proteins in situ by using fusion protein strategies. Applicants refer to Green fluorescent protein: properties, applications and protocols (M. Chalfie, S. Kain, Eds.) Wiley-Liss, 1998, NY; Protein localisation by fluorescence microscopy: a practical approach (VJ Allan, Ed.) Oxford University Press, 2000, NY; Imprint oxford; New York: Oxford University Press, c2000; Oancea, E. et al “Green Fluorescent Protein (GFP)tagged Cysteine-rich Domains from protein Kinase Cas Fluorescent Indications for Diacylglycerol Signalling in Living Cells” The Journal of Cell Biology, Volume 140, No.3, February 9, 1998, pages 485-498.

Multiple cloning site is a special sequence available in all commercially available plasmids, having the property of being cut by sequence specific enzymes. MCSs are normally used to insert genes in order to clone or express foreign genes in a host organism. Applicants refer to J. Sambrook and D. W. Russel, Molecular cloning: a laboratory manual Vol 1-3, Cold Spring Harbor Laboratory Press, 2001, Cold Spring Harbor.

As the efficiency, selectivity and maximum attainable loading of enzymes upon commercially produced surfaces bear strongly on product performance, much effort has been invested to introduce user-friendly, selective, vectorially orienting and easy-to-quantify methods of immobilisation. However there is up to now no process enabling the direct immobilisation of proteins with facility, ease of detection and quantification.

An aim of the present invention is to provide a two-component system, described by a protein encoded by a recombinant plasmid DNA construct and an activated support material, which permits the direct immobilisation and purification of enzymes and proteins with unsurpassed facility and ease of detection.

It is also an aim of the present invention to provide a two-component system, which permits easy visual detection and quantification through light emission.

It is another aim of the present invention to provide a protein encoded by a recombinant plasmid DNA construct permitting to incorporate additional restriction enzyme sites within the multiple cloning site.

It is another aim of the present invention to provide an immobilisation method, which does not require metal ion unlike established purification methods based on His-tag interactions, the interaction between surface and histidine being a direct interaction.

It is another aim of the present invention to provide a technique that can be extended to any protein tag linked to any expressed protein as long as the surface is designed to bear affinity for it.

It is another aim of the present invention to provide a novel recombinant plasmid construct, specialised for expression and directional immobilisation of any fusion protein or enzyme, permitting easy visualisation and better quantification upon surfaces.

It is another aim of the present invention to afford orientation and arraying of enzymes along a surface.

Finally it is an aim of the present invention to provide a large distance separating protein and surface to enable immobilised enzymes to display native-like characteristics, which may prove advantageous in certain cases.

The above aims have been achieved by Applicants invention.

The invention relates to a ircular recombinant plasmid DNA construct encoding a) a protein tag, b) a visual marker protein, and containing c) a multiple cloning site suitable for insertion of an additional gene, characterised in that the gene sequence encoding the protein tag and the visual marker protein are specifically designed and engineered at the DNA level for respectively a) immobilisation purposes and b) visualisation and quantification purposes at the protein level.

The protein tag may be chosen from the group containing lysine (lys), histidine (his), tyrosine (tyr), phenylalanine (phe), arginine (arg), glutamic acid (glu), aspartic acid (asp), glutamate, aspartate, asparagine (asn), glycine (gly), glutamine (gin), alanine (ala), valine (val), tryptophan (trp). The protein tag may be a histidine-tag such as a polyhistidine variant, in particular (6×) histidine.

The visual marker protein may be chosen from the group containing fluorescent or phosphorescent proteins. The fluorescent protein may be chosen from the group containing Green Fluorescent Protein (GFP), Red Fluorescent Protein (RFP), Yellow Fluorescent Protein (YFP) and Blue Fluorescent Protein (BFP) as well as their variants and/or mutants.

The multiple cloning site contains restriction enzyme recognition sites. The restriction enzyme recognition site is chosen from the group containing Sac I, Sal I, Hid IlIl, Eag I, Not I. The construct further contains a frame adapter of variable length between the visual marker and protein tag genes.

The invention also relates to a protein expressed by circular recombinant plasmid DNA construct cited above, wherein in the MCS adjacent to the visual marker, it further contains an additional target protein and in that the tag is suitable to interact directly with appropriate surface pendant groups of a support material. The protein may be a fusion protein.

The invention further relates to the use of the protein, in immobilisation and visualisation of proteins on compatible support material.

One other aspect of the invention is a method for preparing and immobilising a protein on a support material, said method contains the steps of:
a) Engineering at the DNA level, in series a protein tag suitable to interact directly with appropriate surface pendant groups of a support material, a fluorescent marker protein for visualisation and quantification purposes at the protein level and a multiple cloning site suitable for insertion of a target protein to be immobilised,
b) inserting the corresponding gene of the target protein to be immobilised into the multiple cloning site;
c) initiating protein expression.
d) Optionally pre-treating the support material;
e) Incubating the protein and support material together, wherein the protein is immobilised to the support via specific tag-surface interactions;
f) Washing away the non-specific biomolecules;
g) Optionally quantifying the fluorescence of the visual marker protein;
h) Optionally releasing the target protein.

The support material is chosen from the group containing polymers, biopolymers, glass and composites containing silicone dioxides, metals and metal oxides, as well as any combination thereof on the microscopic, mesoscopic or macroscopic length scale.

The support material is preferably chosen from the group containing polymers, silicon dioxides, aluminium oxides, titanium oxides, magnesium oxides, borates, metals and other metal oxides.

The polymers may be chosen from the group containing polyolefins such as polystyrene, polyacrylates, polymethyacrylates, polybutylene, polyvinylalcohol and related derivatives, polyvinylchlorides, polyisoprene, polypropylene, polyphenols, polyamides, polyesters polysulfones, polyethersulfones, polyethersulfides, polyimines, polyethyleneglycols, polypropyleneglycols, polyimides, polycarbonates, polyurethanes,

The polymer surface may be chemically treated to bear various functional groups chosen between carboxyl groups, hydroxyl groups, amino groups, amide groups, ester groups, imide groups, imine groups, mercapto groups, nitro groups, sulfonate groups, phosphate groups, phosphonate groups, cyano groups, sulfone groups, aldehyde groups, epoxide groups, urethane groups, ketone groups, phenolic groups, aromatic groups, alkyl, alkenyl, alkynyl, acyl and aryl groups, silanol groups, silicon oxide groups, siloxane groups, metal hydroxide groups, metal oxide groups, and elemental metals. The support material may be carboxylated polystyrene.

The invention also relates to the immobilised protein construct obtained by the above method, wherein it is covalently or non-covalently bonded to the support material. The immobilised protein construct is non-covalent and yet freely accessible and retained like proteins immobilised in the covalent sense.

Finally the invention relates to the use of the immobilised protein constructs in applications selected from analysis, diagnosis (like in enzyme based diagnostic kits), incubation, storage, sensing, arraying and orienting, catalysis, stabilisation, binding, signal transduction, chemical transformation, implant passivation and surface biocompatibilization, surface activation, purification, detoxification and scavenging.

### FIGURES

FIG. 1 is the Schematics of Experimental: Preparation of a recombinant plasmid DNA construct for immobilisation

FIG. 2 shows the Resultant plasmid

FIG. 3 is a scheme of immobilisation

FIG. 4 shows the Successful Immobilisaton of the GFPimm protein

FIG. 5 is the graphical representation of well fluorescence

FIG. 6 is the infrared spectroscopic profile of the topmost micron layer of native polystyrene;

FIG. 7 is the infrared spectroscopic profile of the topmost micron layer of oxidized polystyrene, implying the formation of surface hydroxyl and carboxylic acid and possibly ketone functionalities.

### DEFINITIONS

Protocol 1 is from QIAGEN® Plasmid Purification Handbook 12/2002 pages 16-20 “Plasmid or Cosmid DNA Purification using QUIAGEN® Plasmid Midi and Maxi Kits”.

Protocol 2 is from QlAquick Spin Handbook 07/2002, pages 23-24 “QIAquick Gel Extraction Kit Protocol using a microcentrifuge”

Protocol 3 is from QlAprep Miniprep Handbook 03/2002, pages 22-25 “QiAprep Spin Miniprep Kit Protocol using a microcentrifuge”.

Protocol 4 is from Version 1 020402 25-0006 of Probond Purification System “A manual of methods for purification of polyhistidine-containing recombinant proteins”

Sequence listing 1 is the GFP Gene

Sequence listing 2 is the pETM-1 I

Sequence listing 3 is the pETM-ADP.

Sequence listing 4 is the pGFPuv

Sequence listing 5 is the pETM-GFP-imm

The term frame adapter refers to the following sequence listing:

| | |
|---|---|
| 1 | GTACGCCATG GGAGGCACGG TACCTTGTG |

The term “native-like activity” refers to the activity of an immobilized enzyme which is not diminished significantly when compared to its activity in solution. “Native” in this case refers to solution phase for the enzyme.

The term “recombinant plasmid DNA construct” refers to an artificially constructed circular DNA, combining genes obtained from different organisms in nature, using molecular biology protocols.

The term “protein encoded by a recombinant plasmid DNA construct” refers to a recombinant protein which is the final product of protein synthesis where the recombinant plasmid DNA construct was used as the template and source of genetic information for the said synthesis.

The term “MAD peptide coding sequence” refers to a gene on the original pETM-11 plasmid, which was excised to modify the plasmid to provide space for insertion of GFP gene as a part of protocols resulting in the invention.

The term “restriction enzyme recognition site” refers to special sequences at which restriction enzymes cut DNA with a high specificity. Each restriction enzyme specifically cuts at its own recognition site on the DNA being engineered.

The term “expression vector or plasmid” refers to a plasmid specialized for high efficiency protein synthesis of a gene inserted in the expression vector's multiple cloning site.

The term “multiple cloning site or MCS” refers to a special sequence designed and artificially constructed on the plasmid, which has a number of restriction enzyme recognition sites together, used to provide open ends for insertion of linear gene fragments in the circular plasmid. Production of open ends is performed by digestion of circular plasmid with restriction enzymes that only cut at their specific recognition sites while combining ends of linear gene and open ends of plasmid is performed by ligation with DNA ligase enzyme.

The term “protein expression” refers to synthesis of proteins using genetic information in the gene for the protein.

The term “DNA level” refers to the type of activity, which is designed and/or performed on the gene before protein synthesis has taken place, but is affecting the protein.

The term “Visual marker protein” refers to a protein (such as GFP), which can be visualised by its fluorescence after it is excited by a characteristic wavelength of light.

The term “immobilisation” refers herein to protein attached to surfaces via a non-covalent interaction. The classic definition of immobilisation refers to covalent and non-covalent methods whereas the classically used terms referring exclusively to non-covalent binding are adsorption or entrapment. The work herein attempts to contrast the facility and generality of immobilisation using the non-covalent tags against the normal methods based upon covalent linkers.

The term “support” refers to any polymeric solid in membrane, particle or wall format, natural or synthetic, porous or nonporous, of organic, inorganic, metallic, or combined composition thereof in the microscopic, mesoscopic or macroscopic length scale, that may or may not be soluble, even transiently under certain conditions, and which bears appropriate surface physico-chemical traits to interact favourably with the fusion protein tag, affording immobilisation. Typical examples of synthetic polymers, out of which composites may also be fashioned, as well as example composites, include, but are not limited to, polyolefins such as polystyrene and related side-chain derivatives such as carboxylated polystyrene, Dowex or chloromethylpolystyrene, polypropylene and side-chain derivatives such as carboxylated polypropylene, polyethylene and related side-chain derivatives, polymethylmethacrylate and related side-chain derivatives such as methyl esters, polyacrylate and side-chain derivatives such as polyacrylamide and polyacrylonitrile, polyethersulfones, polyphenylene, polyphenylenesulfide, polysulfone, graphite and active carbon, polyisobutylene, polyisoprene, polycarbonates, polyphenols such as resol and Novolac, polyesters such as polyalkanoate and polyethyleneteraphthalate, polyamides such as Nylon, polyimides, polyimines, polyvinylalcohols and derivatives, polyethyleneglycol, polyvinylesters, polyethers, polyvinylchloride, polytetrafluoroethylene, polyepoxides, polyurethanes, polychlorotrifluoroethylene, cellulose and nitrocellulose, agarose and related polysaccharides, perfluorinated polymers such as Nafion, silicones, soft glass, borosilicate glass, quartz glass, and other derivatives of silicon dioxide, metal oxides such as iron oxide, titanium dioxide, and aluminum oxide, metal silicates and aluminates such as magnesium silicate and aluminum silicate. Typical examples of natural polymers include, but are not limited to kaolin, diatomaceous earth, sand, volcanic pumas and other inorganics, coal, wood or cellulose, alginic acid, agar, chitin and derivatives, amylose, glycogen, DNA and other polysaccharides, parrafins and triglycerides.

The term “purified” as applied to proteins herein refers to a composition wherein the desired protein comprises at least 80% of the total protein component in the composition. The composition may contain other compounds such as carbohydrates, salts, lipids, solvents and the like without affecting the determination of the percentage purity.

### DETAILED DESCRIPTION OF THE INVENTION

I. Preparation of a Recombinant Plasmid DNA Construct for Immobilisation

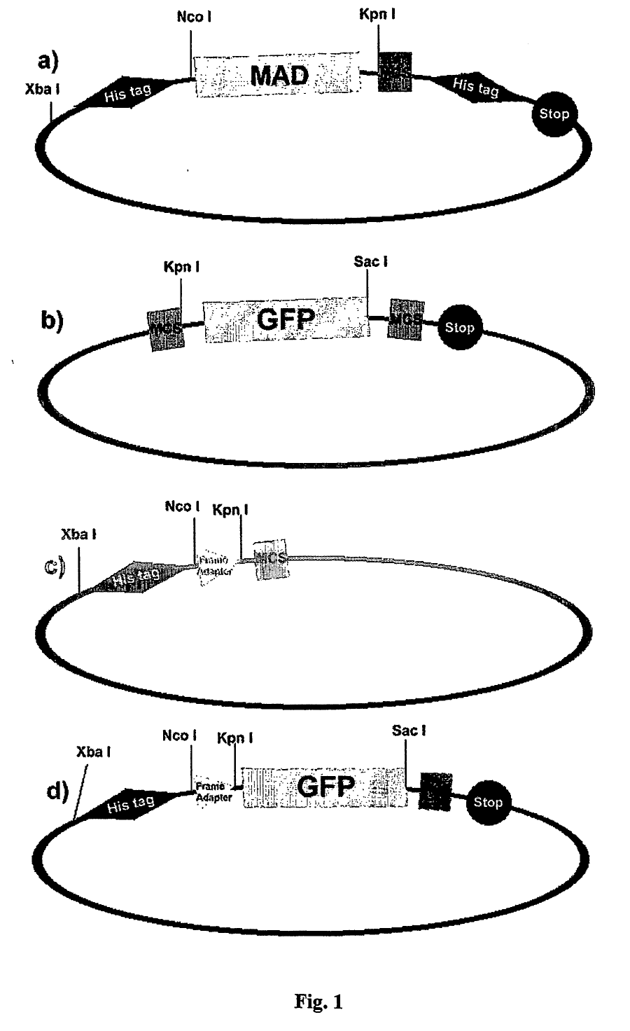

For this preparation reference is made to FIG. 1, wherein a) shows a native vector DNA, that is an protein tag coding plasmid, b) shows a native vector DNA encoding the visual marker protein; c) shows the modified protein tag coding plasmid modified for inserting the visual marker protein in correct frame and d) shows the final recombinant vector specialised for immobilisation of any enzyme or protein.

Multiple adjacent histidine residues incorporated into a protein structure have been applied for the selective purification of recombinant proteins by means of a stepwise process in which the His-tag is recognised, the target protein is immobilised, the non-specific biomolecules are washed away, and the target protein is desorbed. A related process is used herein.

Multiple cloning site (MSC) is a special sequence that has the property of being cut by sequence specific enzymes. MCS are normally used to insert genes in order to clone or express foreign genes in a host organism. In Applicants' system, the multiple cloning site may be used in order to introduce the gene for the enzyme to be immobilised. Another approach may be to introduce additional restriction enzyme sites to MCS in order to enhance space availability and insertion combinations for the gene of the enzyme to be immobilised.

A commercially available, protein tag coding plasmid like the pETM-11 given in Seq. List. 2 is purified and digested to excise MAD protein coding sequence, which is the MAD gene (FIG. 1a). The expression vector (plasmid) pETM-11 in particular was chosen for the reason that its histidine-rich tag could facilitate the subsequent purification and immobilisation of expressed proteins and direct their surface orientation. pETM-11 also bears a cleavable linker gene which is a DNA sequence coding for a small sequence of amino acids that has a specific chemical reactivity allows controlled cleavage by the addition of the appropriate external agent.

On the other hand a frame adapter having the following sequence listing is synthesised. The frame adapter was designed to code for a special sequence of amino acids (Met-Gly-Gly-Thr) forming a high flexibility region in the immobilisation adapter protein.

| | |
|---|---|
| 1 | GTACGCCATG GGAGGCACGG TACCTTGTG |

Then the frame adaptor is inserted into the modified plasmid to give a novel pETM-Adp plasmid like the one given in Seq. List. 3 (FIG. 1c). Bacteria are transformed for amplification of plasmid and the amplified pETM-Adp plasmid is isolated from bacteria and stored.

Then the stored pETM-Adp plasmid is digested to make it ready for another insertion. On the other hand a commercially available native vector DNA like pGFPuv (encoding the visual marker protein GFP) given in Seq. List. 4 is digested to make it ready for insertion (FIG. 1b).

The green fluorescent protein has served to identify the location of other proteins in situ by exploiting fusion protein strategies. Although in Applicants' model GFP variant is used, any fluorescent or phosphorescent protein such as Red FP, Yellow FP, Blue FP and/or their variants and mutants can also be used.

The strategy adopted was to fuse the plasmid to a GFPuv (green fluorescent protein) gene followed by a multiple cloning site. In principle, this multiple cloning site can be used to introduce the gene of any protein, barring size constraints. It would follow that the location and loading of immobilised proteins can be conveniently assessed on the basis of fluorescence emitted by the accompanying adaptor protein.

Then the GFPuv gene is inserted into pETM-Adp to give the novel pETM-GFP-lmm plasmid construct like the one given in Seq. List.5. This is the final recombinant vector specialised for immobilisation of any enzyme of protein. Bacteria are transformed for amplification of plasmid and the amplified pETM-GFP-lmm plasmid is isolated and stored.

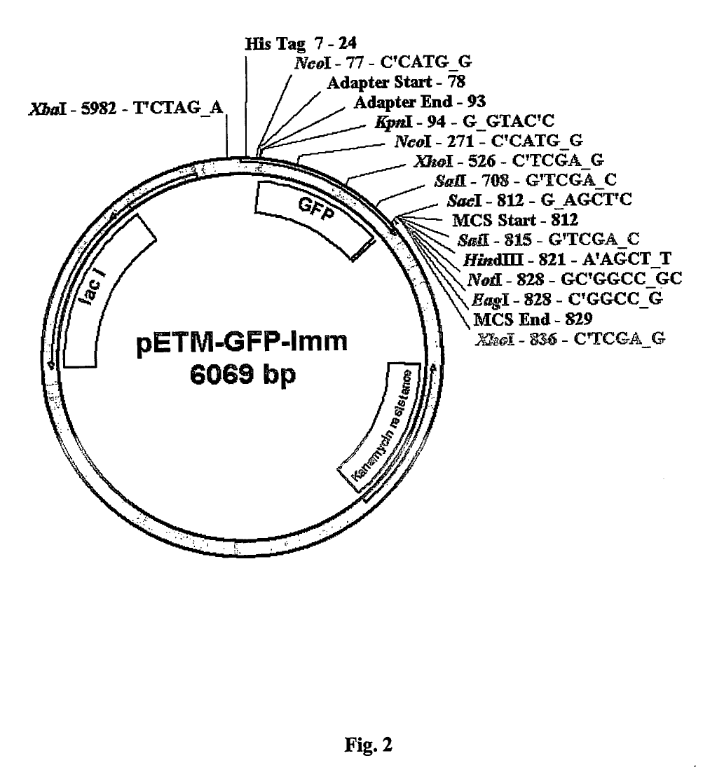

The novel pETM-GFP-lmm plasmid construct does not contain the gene of enzyme to be immobilised but it is designed for another insertion using multiple cloning site after the visual marker protein. (FIG. 2)

FIG. 2 shows the final recombinant vector specialised for immobilisation of any enzyme of protein in that it has a special place preserved for the insertion of new restriction enzyme sites and the insertion of gene of the enzyme or any protein to be immobilised.

The production facilities of this vector as well as the affinity purification techniques of proteins are established and cost-effective. More importantly, the advantages stated herein are not in any way compromised by disadvantages which are exclusive to this particular process.

II. Immobilisation of Protein Product of Recombinant Plasmid DNA on a Compatible Polymer Surface

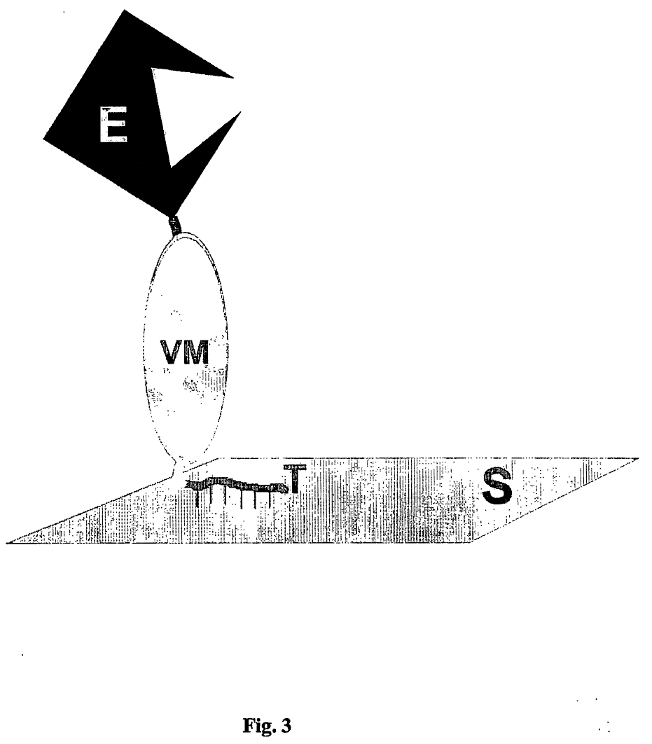

For this immobilisation reference is made to FIG. 3 wherein an enzyme or protein (E) containing a visual marker protein (VM) is bonded through its tag (T) to the surface of a compatible support material (S).

Bacterial culture of the novel plasmid —pETM-GFP-lmm—bearing expression strains are grown. On the other hand bacterial culture of visual marker protein coding plasmid—pGFPuv—bearing expression strains are grown. Then both bacterial cultures were lysed and GFPuv and protein tag containing GFPimm proteins are purified. For both proteins the sodium concentration is lowered and imidazole is removed by dialysis in preparation of surface binding.

The surface of the compatible support material is prepared. In this particular case the polystyrene surface is oxidised in preparation for immobilisation. Polymer surfaces have been tailored using chemical methods to bear various functional groups. The carboxyl group in particular has been applied in the reversible/irreversible surface immobilisation of positively charged species as well as hydrogen bonding species.

GFPimm protein is bound to the surface of a compatible support material. Said compatible support material can be a polymer, a biopolymer, a glass or a metal. A prefabricated surface and complementary tag showing good interaction with the surface is used.

The green fluorescent protein has served to identify the location of other proteins in situ by exploiting fusion protein strategies. Although in Applicants' model a GFP variant is used, any fluorescent or phosphorescent protein such as Red FP, Yellow FP, Blue FP and their variants and/or mutants can be used. These fluorescent proteins can be used even in a combined manner that the different enzymes of the same enzymatic pathway can be immobilised and easily quantified on different regions of the same polymeric support.

Applicants' invention permits to avoid all disadvantages of immobilisation technology while benefiting from the advantages of said technology. In Applicants' system, polyhistidine tag is used for ionic interaction between their positive charges and negative charge loaded on polyrmeric surface, the difference being that the interaction between surface and tag is direct as opposed to mediated by a coordinating metal ion.

Unlike established purification methods based on His-tag interactions, Applicants' immobilisation method does not require metal ion, the interaction between surface and histidine being a direct interaction.

Furthermore, Applicants' technique can be extended to any protein tag as long as the surface is designed to bear affinity for it. Similar approach may be applied to other protein tags with appropriate tailored surfaces provided that the tag and the surface have affinity to each other.

Measurements are made to show the binding. The distance separating protein and surface being large in comparison to many immobilisation methods, immobilised enzymes in particular are anticipated to display native-like characteristics.

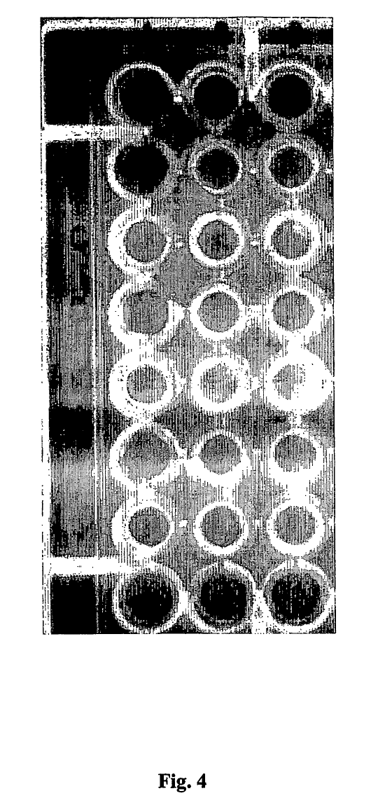

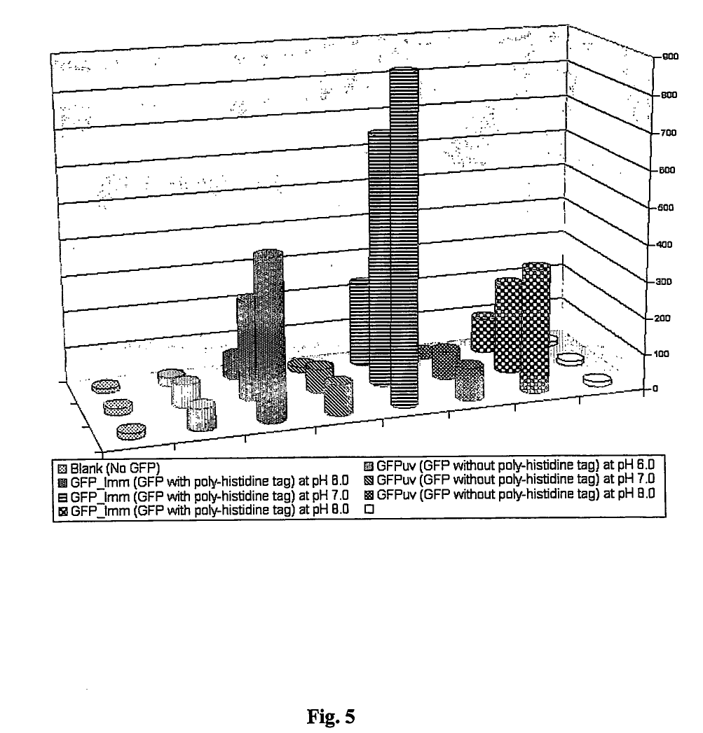

It is made reference to FIGS. 4 and 5 wherein FIG. 4 shows a photo with an integration time of 190 ms and FIG. 5 shows its fluorescence in relative fluorescence units.

Before taking the photo of FIG. 4, GFPimm and GFPuv of approximately same concentration were incubated during 2 hours and washed 3 times with the appropriate buffer in which binding reaction occurred being different in that rinsing buffer does not contain any form of GFP. After 3 washes with buffer, 25mM sodium phosphate, 250 mM NaCI solution (the pH was appropriately adjusted with NaOH), the wells were dried by inverting the well over paper towels and tapping the backs repeatedly.

TABLE
96 Well Plate Assay using Gemini
XS fluorometer-dry well readings.

| | 0MAPS | 1MAPS | 2MAPS | empty |
|---|---|---|---|---|
| NoGFP | 12.02 | 19.511 | 17.073 | 13.635 |
| GFPpH6 | 23.96 | 64.387 | 62.106 | 20.444 |
| GFPHispH6 | 63.792 | 274.97 | 443.267 | 76.069 |
| GFPpH7 | 15.509 | 67.949 | 85.149 | 22.401 |
| GFPHispH7 | 233.672 | 690.85 | 888.078 | 145.186 |
| GFPpH8 | 16.176 | 78.776 | 89.982 | 23.807 |
| GFPHispH8 | 99.532 | 250.865 | 344.894 | 50.509 |
| EMPTY | 12.462 | 13.748 | 13.114 | 13.251 |

The data of the above table and the Figures verify that GFPimm was selectively immobilised on carboxylated polystyrene at pH 6, 7 and 8 whereas native GFPuv was not similarly retained following repeated washings. Moreover, the data indicates that a non-specific interaction, albeit small in comparison, is still present between GFPimm and native polystyrene. For this reason, the possibility of introducing tyrosine or tryptophan tags is also noteworthy, as they can in principle also bind to the aromatic surface via aromatic-aromatic stacking.

Unlike established purification methods based on His-ag interactions, a difference with the technology herein is that no metal ion is required to enable the immobilisation event. The interaction between surface and histidine is a direct interaction between surface-pendent carboxyl groups and imidazole moieties of the his-tag in the protein.

The reversal of binding is facile in simple species that bear few positive charges. In systems involving much larger, multi-charged molecules such as proteins, the biomolecule is often immobilised by its own inertia. In these situations, proteins may in fact be irreversibly immobilised even though no formal covalent bond is created. This relationship is particularly relevant in the short term but it can revert to a reversible association in time.

Since the distance separating protein and surface is large in comparison to many immobilisation methods, immobilised enzymes in particular are anticipated to display native-like characteristics, which may prove advantageous in certain cases.

Examples of alternative interactions include hydrogen-bonding, hydrophobic interactions such as base stacking and other ionic bondings such as positively charged ammonium sulfates and negatively charged amino acids.

Indeed examples of such interactions have been established, however, the notable difference with the proposed technology herein is that the tag is specifically designed and engineered on the protein at the DNA level.

Fusion proteins potentially represent very common products of this technology. Proteins fused to GFPimm will feature a facile means of visualisation in addition to a facile means of purification. In applicants system GFP is used in order to visualise final location and quantify the immobilised enzyme that is fused to the fluorescent protein genetically.

Applicants' novel recombinant plasmid technology specialised for fusion protein expression and immobilisation, permits easy visualisation and better quantification upon surfaces. Fusion proteins potentially represent very common products of this technology. Proteins fused to the marker will feature a facile means of visualisation in addition to a facile means of purification.

The immobilisation strategy also required that a substrate surface respond selectively to the his-tag. To this end, Applicants devised several surfaces bearing linkers that could interact covalently or electrostatically with the imidazole functional group.

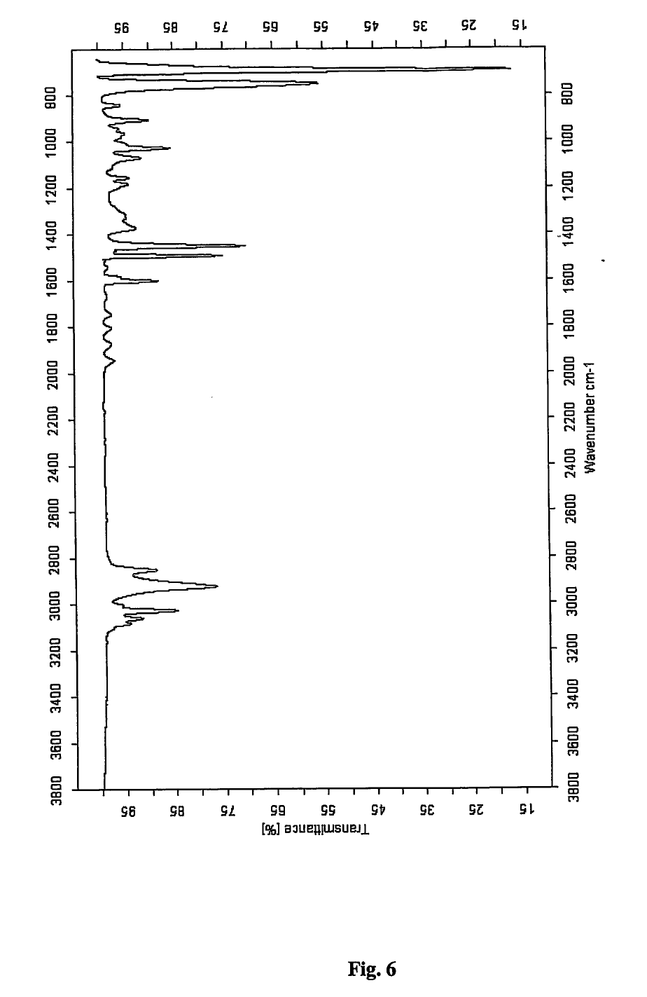

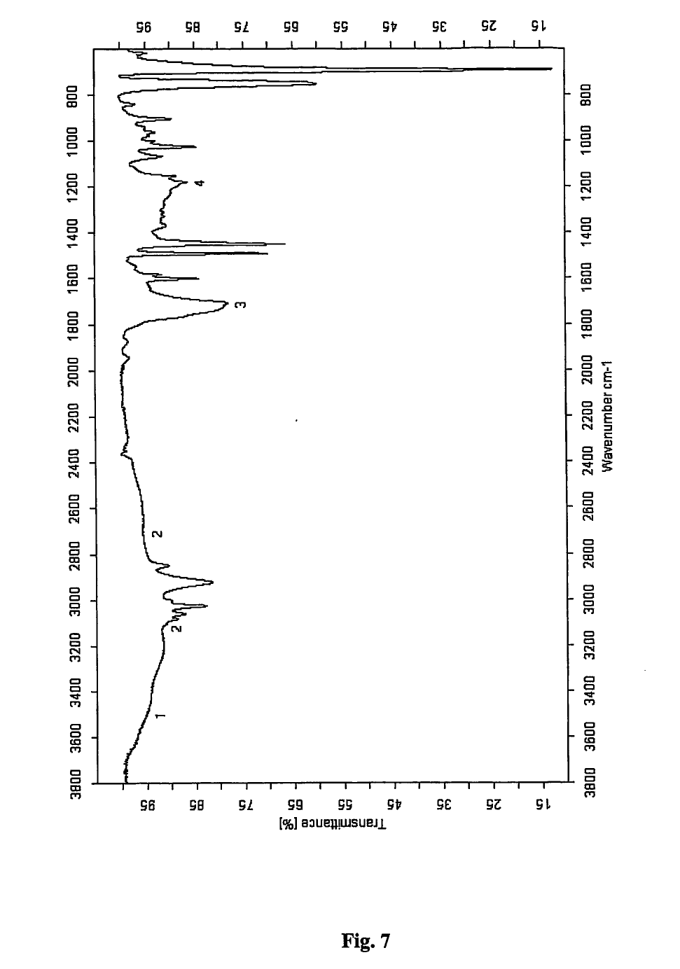

Now it is made reference to FIGS. 6 and 7 wherein FIG. 6 is the infrared spectroscopic profile of the topmost micron layer of native polystyrene and FIG. 7 is the infrared spectroscopic profile of the topmost micron layer of oxidized polystyrene, showing the formation of surface hydroxyl and carboxylic acid and possibly ketone functionalities introduced for interacting with protein tag in the designed protein construct.

In FIG. 7, 1 stands for H—O stretch of alcohol; 2: stands for H—O stretch of carboxylic acid; 3: stands for C═O stretch of carboxylic acid and ketone; 4: stands for C—O stretch of alcohol and carboxylic acid.

In summary Applicants' invention includes following advantages: 1) the flexibility to incorporate enzymes or any proteins or additional restriction enzyme sites within the multiple cloning site, 2) the flexibility to incorporate any amino-acid tags, 3) the flexibility to put distance between the surface and the protein, improving the likelihood that native-like activity of anchored proteins is retained; 4) the possibility to achieve spatial orientation of proteins and permit their arraying along a surface, 5) to permit easy visual detection and quantification through light emission and 6) to avoid exposing protein to potentially harmful chemical modifications.

Current work is directed at optimising the conditions of immobilisation, quantifying the bound protein, and characterising its function.

The following example is given for illustration purposes and does not restrict or limit whatsoever the invention.

### EXAMPLE

### The Protocols used in the Example:

QIAGEN Plasmid Purification Handbook 12/2002 Plasmid or Cosmid DNA Purification using QIAGEN Plasmid Midi and Maxi kits, pages 16-20.

QIAquick Spin Handbook 07/2002, “QlAquick Gel Extraction Kit Protocol using a microcentrifuge”, pages 23-24

QlAprep Miniprep Handbook 03/2002 “QIAprep Spin Miniprep Kit Protocol using a microcentrifuge”, pages 22-25.

Protocol for ProBond Purification System, A manual of methods for purification of polyhistidine-containing recombinant proteins, Catalog no. K850-01, K851-01, K852-01, K853-01, K854-01 from www.invitrogen.com tech_serviceinvitrogen.com

### Plasmids, Enzymes, Chemicals and Experimental Kits and Buffers Used in the Example:

| | |
|---|---|
| Acetic Acid | Riedel-de Haén, Catalog#: 27225, |
| | CAS 64-19-7 |
| Acrylamide: | Sigma, Catalog#: A3699, CAS 79-06-1 and |
| Bis-acrylamide | 110-26-9 |
| Agarose | Sigma, Catalog#: A-6138, CAS9012-36-6 |
| Ammonium | Carlo-Erba, Italy, Catalog#: 420627, |
| persulfate | |
| Ampicillin | Sigma, Catalog#: A9518 |
| Boric Acid | Riedel-de Haén, Catalog#: 11607 |
| BSA (Bovine | Promega, supplied with Kpn I, Nco I and Sac I |
| Serum Albumin) | |
| (10× concentrated) | |
| Distilled water, | Millipore, MilliQ Academic, France |
| sterile, | |
| MilliQ filtered | |
| EDTA | Riedel-de Haén, Catalog#: 27248 |
| Ethanol | Riedel-de Haén, Catalog#: 32221 |
| Ethidium Bromide | Merck, Catalog#: OCO28942 |
| Frame Adapter | Synthesized by Seqlab, Germany (design |
| | E. Sahin) |
| Glycerol | Riedel-de Haén, Catalog#: 15523 |
| HCl | Merck, Catalog#: 100314 |
| Imidazole | Sigma, Catalog#: I-2399 |
| IPTG | Promega, Catalog#: V39517 |
| Isopropanol | Riedel-de Haén, Catalog#: 24137 |
| Kanamycin | Sigma, Catalog#: K4000 |
| Kpn I | Promega, Catalog#: R634A |
| Liquid nitrogen | Karbogaz, Turkey |
| Luria Agar | Sigma, Catalog#: L-3147 |
| Luria Broth | Sigma, Catalog#: L-3022 |
| Lysozyme | Merck, Catalog#: 1.05281 |
| Mass Ruler | Fermentas, Catalog#: #SM0393 High Range |
| DNA Ladder | |
| Mass Ruler | Fermentas, Catalog#: #SM0383 Low Range |
| DNA Ladder | |
| 2-Mercaptoethanol | Aldrich Chemical Company, M370-1 |
| Micro-Spin ™ | Amersham Biosciences, Catalog#: 27-5325-01 |
| G-25 Columns | |
| Multi-Core ™ | Promega, supplied with Kpn I, Nco I and Sac I |
| Buffer (10×) | |
| NaCl | Riedel-de Haén, Catalog#: 13423 |
| NaH₂PO₄ | Merck, Catalog#: 106370 |
| NaOH | Merck, Catalog#: 106462 |
| Native | Composed of Na₃PO₄ and NaCl. |
| Purification | Preparation is given in Protocol 4 section. |
| Buffer (5×) | |
| for ProBond | |
| Nco I | Promega, Catalog#: R651F |
| Nickel Chloride | Merck, 806722 |
| pETM-11 | European Molecular Biology Laboratories, |
| | Germany |
| pGFPuv | Clontech, Germany |
| Polystyrene | TPP, TP92696 |
| 96-well plate | |
| Polystyrene | TPP, TP93100 |
| culture plate | |
| ProBond ™ | Invitrogen, Catalog#: 46-0019 |
| resin | |
| Qiagen Midi- | Qiagen, Catalog#: 12145 |
| prep Plasmid | (All buffers in protocols are included) |
| Purification Kit | |
| Qiagen Mini- | Qiagen, Catalog#: 27106 |
| prep plasmid | (All buffers in protocols are included) |
| isolation kit | |
| Qiagen | Qiagen, Catalog#: 28706 |
| QIAquick | (All buffers in protocols are included) |
| Gel | |
| Extraction Kit | |
| RNAase | Qiagen, supplied with Qiagen Plasmid |
| | Isol. Kits |
| Sac I | Promega, Catalog#: R606A |
| Sodium | Sigma, Catalog#: L-4390 |
| Dodecyl | |
| Sulphate | |
| T4 DNA Ligase | Promega, supplied with T4 DNA Ligase |
| Buffer (10×) | |
| T4 DNA Ligase | Promega, Catalog#: M180B |
| TEMED | Sigma, Catalog#: T-7029 |
| Tris | Fluka, Catalog#: 93349 |
| Tris-Acetate- | Composed of Tris, acetic acid and EDTA. |
| EDTA Buffer | For preparation see J. Sambrook and |
| | D. W. Russel, Molecular cloning: a |
| | laboratory manual Vol 1-3, |
| | Cold Spring Harbor Laboratory Press, 2001, |
| | Cold Spring Harbor. |
| Tris-Borate- | Composed of Tris, acetic acid and EDTA. |
| EDTA Buffer | For preparation see J. Sambrook and |
| | D. W. Russel, Molecular cloning: a |
| | laboratory manual Vol 1-3, |
| | Cold Spring Harbor Laboratory Press, 2001, |
| | Cold Spring Harbor. |
| Tris-HCl | Composed of Tris and HCl. Tris is dissolved to |
| | a given molarity and pH is adjusted with HCl. |
| Urea | Merck, Catalog#: 1.12007.2500 |

### Instruments and Equipment Used in the Example:

| | |
|---|---|
| Autoclave: | Hirayama, Hiclave HV-110, JAPAN |
| Balance: | Sartorius, BP221S, GERMANY |
| | Schimadzu, Libror EB-3200 HU, JAPAN |
| Centrifuge: | Eppendorf, 5415D, GERMANY |
| | Hitachi, Sorvall RC5C Plus, USA |
| Deepfreeze: | −80° C., Kendro Lab. Prod. |
| | Heraeus Hfu486 Basic, Germany |
| | −20° C., Bosch, TURKEY |
| Dialysis tubes | CelluSep T3, Membrane Filtration |
| | Products Inc., USA |
| Distilled Water: | Millipore, Elix-S, FRANCE |
| | Millipore, MilliQ Academic, FRANCE |
| Electrophoresis: | Biogen Inc., USA |
| | Biorad Inc., USA |
| Filter membranes | 0.2 μm filter: Millipore, Catalog# |
| | GNWP04700, USA |
| | 0.45 μm filter: Millipore, Catalog# |
| | HNWP04700, USA |
| Fluorometer: | SPECTRAmax GEMINI XS, Molecular |
| | Devices Corporation, USA |
| FPLC | ÄKTA FPLC, Amersham Biosciences |
| FPLC Column | MonoQ Ion Exchange Chromatography Column |
| | (10/10), |
| | Amersham Biosciences |
| Gel Documentation: | UVITEC, UVIdoc Gel Documentation |
| | System, UK |
| | Biorad, UV-Transilluminator 2000, USA |
| Heater Block: | Bioblock Scientific, FRANCE |
| Ice Machine: | Scotsman Inc., AF20, USA |
| Incubator: | Memmert, Modell 300, GERMANY |
| | Memmert, Modell 600, GERMANY |
| FT Infrared | Equinox 55 TGA-IR, Bruker Optics Inc. |
| Spectrometer: | with Attenuated Total Refl. Accessory |
| Laminar Flow: | Kendro Lab. Prod., Heraeus, HeraSafe |
| | HS12, GERMANY |
| Magnetic Stirrer: | VELP Scientifica, ARE Heating Magnetic |
| | Stirrer, ITALY |
| | VELP Scientifica, Microstirrer, ITALY |
| Microliter Pipette: | Gilson, Pipetman, FRANCE |
| | Mettler Toledo, Volumate, USA |
| Microwave Oven: | Bosch, TURKEY |
| pH meter: | WTW, pH540 GLP MultiCal ®, GERMANY |
| Polystyrene | TPP, SWITZERLAND |
| 96-well plate | |
| Power Supply: | Biorad, PowerPac 300, USA |
| | Wealtec, Elite 300, USA |
| Refrigerator | +4° C., Bosch, TURKEY |
| Shaker: | Forma Scientific, Orbital Shaker 4520, USA |
| | GFL, Shaker 3011, USA |
| | New Brunswick Sci., Innova ™ 4330, USA |
| Sonicator | Vibracell 75043, Bioblock Scientific, FRANCE |
| Spectrophotometer: | Schimadzu, UV-1208, JAPAN |
| | Schimadzu, UV-3150, JAPAN |
| | Secoman, Anthelie Advanced, ITALY |
| Speed Vacuum: | Savant, Speed Vac ® Plus Sc100A, USA |
| | Savant, Refrigerated Vapor Trap RVT |
| | 400, USA |
| Thermocycler: | Eppendorf, Mastercycler Gradient, GERMANY |
| Vacuum: | Heto, MasterJet Sue 300Q, DENMARK |
| Vortex: | Velp Scientifica, ITALY |

### Step 1: Preparation of Vector for Ligation:

pETM11 vector originally has a protein (MAD) coding sequence which is removed to modify the vector for using with larger inserts. For this purpose, enzymatic digestion of purified pETM-11 vector was performed using Kpnl and Ncol restriction enzymes.

A: Purification of pETM-11:

XL1-Blue E.coli strain containing pETM-1 1 plasmid was grown in 50 mL Luria Broth with 50 μg/mL final concentration of Kanamycin at 37° C., at 300 rpm overnight. Overnight grown cultures are used for isolation of pETM-11 plasmid by using Protocol 1. Quantification is done by spectrophotometric absorbance measurement at 260 nm by using the formula: [DNA]=[Abs]×50 ng/μl. The measurement value is verified with gel electrophoresis results.

B: Digestion of pETM-11 with Kpn I and Nco I:

56 μl pETM-11 vector (˜5.6 μg)
8 μl Multi-Core™ buffer system
8 μl BSA
4 μl Kpn I
4 μl Nco I

3 hours incubation was performed at 37° C., digestion was verified with agarose (1% in Tris-Acetate-EDTA Buffer) gel electrophoresis.

MAD coding sequence is removed from pETM-11 by agarose (1% in Tris-Acetate-EDTA Buffer) gel electrophoresis following double digestion with Kpn I and Nco I. Larger fragment (pETM-11 without MAD) was isolated by using Protocol 2. Quantity estimation was done by agarose gel electrophoresis analysis.

Step 2: Preparation of Frame Adapter for Ligation:

The adapter was digested at both sides by Kpn I and Nco I restriction enzymes in six different Eppendorf tubes in parallel, by using the following protocol:
2 μl Adapter (0.025 nmol/μl)
4 μl Multi-Core™ buffer system
6μl BSA
2 μl Kpn I
2 μl Nco I
24 μl dH₂O (MilliQ filtered, sterile)

Overnight incubation was performed at 37° C., 6 tubes of digests were combined together and desalted by passing two times through Micro-Spin™ G-25 Columns. Digestion verification and quantity estimation is performed by polyacrylamide urea gel electrophoresis (3 mL 30%:0.8% —Acrylamide:Bis-acrylamide; 500 μl 10×TBE; 2.1 gr urea; 1.5 mL dH₂O; 45μl APS; 3 μl TEMED).

| | | |
|---|---|---|
| Frame adapter | 1 | GTACGCCATG GGAGGCACGG TACCTTGTG |

Step 3: Ligation:

“pETM-11 without MAD” was ligated with “frame adapter” in order to prepare the vector for insertion of GFP within the right frame of protein synthesis. A wide range of different concentration ratios were applied in order to optimise ligation in this specific case where insert is ˜0.1% of the vector. The following protocol was used:

| PETM-11 + Adapter | | pETM-11 + Adapter | | PETM-11 + Adapter | | Self Ligation | |
|---|---|---|---|---|---|---|---|
| (1:0.2 (v:v)) | | (1:0.8 (v:v)) | | (1:10 (v:v)) | | (control) | |
| 1 | μl pETM-11 | 1 | μl pETM-11 | 1 | μl pETM-11 | 1 | μl pETM-11 |
| 0.2 | μl Adapter | 0.8 | μl Adapter | 10 | μl Adapter | 2 | μl Ligation buffer |
| 2 | μl Ligation buffer | 2 | μl Ligation buffer | 2 | μl Ligation buffer | 1 | μl Ligase |
| 1 | μl Ligase | 1 | μl Ligase | 1 | μl Ligase | 16 | μl dH₂0 |
| 15.8 | μl dH₂0 | 15.2 | μl dH₂0 | 6 | μl dH₂0 | | |

Ligations were performed at 25° C. overnight.

Step 4: Transformation:

50 μl competent E. coli XL1-Blue cloning strain was added on overnight ligation mixes.
Competent cell added ligation mixes were incubated 20 min on ice.
Tubes were incubated at 42° C. heater block for 3 minutes.
900 μl LB (without Kanamycin) was added to each ligation mix.
LB added transformation mixes are incubated for 1.5 hours at 37° C. with 190 rpm shaking.
100 μl and 400 μl aliquots were spread on Luria Agar+Kanamycin plates and the plates were incubated at 37° C. incubator overnight.
   Step 5: Colony Check for Presence of Insert:

5 colonies were picked one by one and grown overnight in 5 mL LB+ Kanamycin cultures individually at 37° C. with 300 rpm shaking. Next day, plasmid isolation was performed by application of Protocol 3 to each overnight culture. Each isolated plasmid was digested by either Kpn I or Nco I under the following conditions:
5 μl mini-prep isolated plasmid
1 μl Multi-Core™ buffer system
1 μl BSA
0.5 μl Kpn I OR Nco I for each mini-prep isolated plasmid
2.5 μI dH₂O (MilliQ filtered, sterile)
Incubation for 3 hours at 37° C.

Digestion efficiency and presence of vector plasmid were detected by agarose (1% in Tris-Acetate-EDTA Buffer) gel electrophoresis analysis in which uncut and cut original (unmodified) pETM-11 were also used as standards. The vector was quantified by spectroscopic analysis at UV-visible wavelength range.

Step 5a-80 Stock Preparation:

After the presence of correct plasmids was verified with agarose gel electrophoresis, glycerol stocks of plasmid bearing colonies were prepared by the following protocol:
To 850 μl overnight grown bacterial culture, 150 μl sterile glycerol was added.
Mixed by vortexing.
Frozen in liquid nitrogen.
Kept at −80° C.
   Step 6- Preparation of adapter bearing vector (pETM-Adp) for ligation:

pETM-Adp was prepared for ligation with GFP gene by enzymatic digestion by Kpn I and Sac I by using the following protocol:
37 μl vector (˜6 μg)
6 μl Multi-Core™ buffer system
6 μl BSA
2.5 μl Kpn I
2.5 μl Sac I
6 μl dH₂O

3 hours incubation was performed at 37° C, digestion was verified with agarose (1% in Tris-Acetate-EDTA Buffer) gel electrophoresis.

The result of enzymatic reaction was evaluated qualitatively and quantitatively by using agarose (1% in Tris-Acetate-EDTA Buffer) gel electrophoresis. Double digested pETM-Adp was isolated by Protocol 2 and isolated linear pETM-Adp was quantified by agarose (1% in Tris-Acetate-EDTA Buffer) gel electrophoresis.

Step 7- Preparation of Insert for Ligation:

GFP coding gene was provided by removing the gene from pGFPuv vector by Kpn I and Sac I double digestion.
56 μl pGFPuv vector (˜5.5 μg)
8 μl Multi-Core™ buffer system
8 μl BSA
4 μl Kpn I
4 μl Sac I

3 hours incubation was performed at 37° C., quality of digestion was verified and quantity of DNA was estimated by using agarose (1% in Tris-Acetate-EDTA Buffer) gel electrophoresis.

Step 8- Ligation:

Linearized (Kpn I and Sac / double digested) pETM-Adp vector and GFP insert (Kpn I and Sac I double digested) were ligated using the following protocol with different concentration ratios between the vector and insert:

| pETM-Adp + | | PETM-Adp + | | pETM-Adp + | | pETM-Adp + | | pETM-Adp | |
|---|---|---|---|---|---|---|---|---|---|
| GFP 1:1 (v:v) | | GFP 1:12 (v:v) | | GFP 2:0.2 (v:v) | | GFP 2:2 (v:v) | | Self ligation | |
| 1 | μl pETM-Adp | 1 | μl pETM-Adp | 1 | μl pETM-Adp | 2 | μl pETM-Adp | 1 | μl pETM-Adp |
| 1 | μl GFP | 12 | μl GFP | 0.2 | μl GFP | 2 | μl GFP | 2 | μl Ligase Buffer |
| 2 | μl Ligase Buffer | 2 | μl Ligase Buffer | 2 | μl Ligase Buffer | 2 | μl Ligase Buffer | 1 | μl Ligase |
| 1 | μl Ligase | 1 | μl Ligase | 1 | μl Ligase | 1 | μl Ligase | 16 | μl dH₂O |
| 15 | μl dH₂O | 4 | μl dH₂O | 14.8 | μl dH₂O | 13 | μl dH₂O | | |

Ligations were performed by incubating above ligation mixes at 25° C. overnight.

Step 9- Transformation:

50 μl competent E. coli XL1-Blue cloning strain was added on overnight pETM-Adp +GFP 1:12; 2:0.2; 2:2; self lig. ligation mixes. 50 μl competent E. coli BL21-DE3 expression strain was added on overnight pETM-Adp +GFP 1:1. In addition to above indicated tubes, one transformation control (1 μl uncut pETM-Adp, 19 μl dH₂O), one negative control (50 μl untransformed BL21-DE3) and one positive control (50 μl untransformed BL21-DE3) were prepared in different tubes for spreading on different media.
Competent cell added ligation mixes were incubated 20 min on ice.
Tubes were incubated at 42° C. heat block for 3 minutes.
900 μl LB (without Kanamycin) was added to each ligation mix.
LB added transformation mixes are incubated for 1.5 hours at 37° C. with 190 rpm shaking.
100 μl and 400 μl aliquots of pETM-Adp +GFP 1:12; 2:0.2; 2:2; self lig.; negative control and transformation control tube contents were spread on Luria Agar+Kanamycin plates, pETM-Adp +GFP 1:1 tube content was spread on a plate half of which was enriched with 1 mM IPTG before the colony spreading step;

positive control was spread on Luria Agar plate and the plates were incubated overnight at 37° C. incubator.

Step 10: Colony Check for Presence of Insert:

Colonies bearing functional GFP gene was selected by checking fluorescence under UV light source. 3 colonies were picked from both XL1-Blue strain and BL21-DE3 strain containing plates. The picked colonies were grown overnight in 5 mL LB+Kanamycin cultures individually at 37° C. with 300 rpm shaking.

Next day, pelleted bacterial cultures were photographed under UV for fluorescence; plasmid isolation was performed by application of Protocol 3 to each overnight culture. Each isolated plasmid was digested by both Sac I and Nco I under the following conditions:
3 μl mini-prep isolated plasmid
1 μl Multi-Core™ buffer system
1 μl BSA
0.5 μl Sac I
0.5 μl Nco I
4 μl dH₂O (MilliQ filtered, sterile)
Incubation for 3 hours at 37° C.

Presence of vector plasmid and presence of insert were detected by agarose (1% in Tris-Acetate-EDTA Buffer) gel electrophoresis analysis and quantified by spectroscopic analysis in UV-visible wavelength range.

Step 10a-80 Stock Preparation:

After the presence of plasmids with inserts were verified with agarose gel electrophoresis, glycerol stocks of plasmid bearing colonies were prepared by the following protocol:
To 850 μl overnight grown bacterial culture, 150 μl sterile glycerol was added.
Mixed by vortexing.
Frozen in liquid nitrogen.
Kept at 80° C.
   Step 11-Expression (Induction of GFP and GFPimm Synthesis):

Starter cultures of GFPuv and GFPimm bearing BL21-DE3 expression strains were grown in 5 mL LB+Ampicillin (0.1 mg/mL Amp.) and LB+Kanamycin (0.05 mg/mL Kan.) respectively for 8 hours at 37° C. with 300 rpm shaking.

Absorbance measurements of cultures were performed at 600 nm and starter cultures were diluted to 0.02 OD in 2×250 mL LB+Ampicillin (0.1 mg/mL Amp.) and LB+Kanamycin (0.05 mg/mL Kan.) in 2>1000 mL Erlenmayer flasks. The cultures were grown overnight at 37° C. with 300 rpm shaking.

Step 12-Purification of GFPimm Protein Construct:

Purification of GFPimm construct was performed by using Protocol 4 with the exception in scaling up so that 40 mL 1× Native binding buffer was used to resuspend bacterial pellet. Isolated GFP was quantified using spectroscopic analysis for 397 nm wavelength which is characteristic local absorption maximum of GFP. Quantification was verified by Sodium Dodecyl Sulphate—Polyacrylamide gel electrophoresis results.

Step 13-Purification of GFPuv Protein:

40 mL 30 mM Tris-HCI, pH 7.5 +Lysozyme (1 mg/mL) was used to resuspend pelleted bacteria. The bacteria suspension was incubated for 30 min. on ice, then sonicated for 4 min with 10(sec):10(sec) pulse:cooling steps on ice.

Cell debris was precipitated by centrifugation at 17000g for 15 min at 4° C.

Soluble phase was filtered through 0.45 and 0.22 μm filters respectively. Filtered solution was applied to MonoQ ion exchange column with a linear salt concentration gradient from degassed, 0.22 μm filtered, 30 mM Tris-HCI, pH 7.5 to degassed, 0.22 μm filtered, 30 mM Tris-HCI, 1 M NaCl pH 7.5 with 0.5 mL/min flow rate.

Isolated GFP was quantified using spectroscopic analysis for 397 nm wavelength which is characteristic local absorption maximum of GFP. Quantification was verified by Sodium Dodecyl Sulphate - Polyacrylamide gel electrophoresis results.

Step 14-Lowering Na* Concentration for Binding Preparation:

GFPuv and GFPimm proteins were dialysed against 25mM Na₃PO₄; 250 mM NaCI with different pH values (pH 6.0, pH 7.0, pH 8.0) for 6 hours at 4° C. GFP samples were then centrifuged in 2 mL Eppendorf tubes and supernatants were taken in clean Eppendorf tubes. Supematants were re-quantified for GFP content using spectroscopic analysis and SDS-Polyacrylamide gel electrophoresis.

Step 15-Surface Preparation:

2 M and 3 M ammonium persulfate solutions were prepared (for 3 M APS preparation, solution must be heated to 45° C.)

Polystyrene 96 well plate was filled 2/3 of its well capacity providing lanes for dH₂O, 2M APS and 3M APS. The 96 well plate was tightly closed and incubated for 24 hours at 70° C. The plate was rinsed with dH₂O and dried in 70° C.

Note—Infrared spectroscopic evidence given in FIG. 6 verified the formation of carboxylic acid functional groups.

Step 16-Binding to Surface:

After verifying that impurity of GFPuv and GFPimm proteins are comparable, their concentration was equalized by diluting concentrated sample considering data from spectroscopic analysis.

Fluorescence of empty plate was measured by Molecular Devices: SpectraMax GeminiXS Fluorometer.
100 μl of GFPuv and GFPimm containing samples were loaded in wells of 96 well plate in an order from pH 6.0 to pH 8.0. Binding was performed with appropriate buffer with the pH of the sample.

Binding was performed by 2 hours incubation at room temperature.

| Blank for Background | dH₂O | 2M APS mod. | 3M APS mod. |
|---|---|---|---|
| GFPuv at pH 6 | X | X | X |
| GFPimm at pH 6 | X | X | X |
| GFPuv at pH 7 | X | X | X |
| GFPimm at pH 7 | X | X | X |
| GFPuv at pH 8 | X | X | X |
| GFPimm at pH 8 | X | X | X |

Step 17-Measurements:
Samples were removed from wells of plate and the plate was rinsed 3 times with pipetting 200 μl appropriate buffer for each well.
All the liquid was removed from the wells.
Fluorescence was measured when the plate was dry.
The wells were filled with 100 μl of appropriate binding buffer and fluorescence measurements were repeated.

Photographs of dry and wet cases for GFP bound plates were taken under UV light.

Life span of the proposed technology is difficult to predict, but it is undeniable that molecular biology has become an irrevocable part of the basic sciences and therefore a potential market for protein products is unrelenting. Areas of use or application may be analysis, incubation, storage, catalysis, stabilisation, binding, purification, scavenging and immobilisation of bio-polymers.

Any industry based on protein expression, protein technology, medicine, or specialty plastics and glasses could realise profit following implementation of this process.

Applicants are also exploring secondary applications that might exploit the physico-chemical properties of this system.

### REFERENCES

Methods in Enzymology: Immobilised Enzymes and Cells, Part B. Vol. 135 (K. Mosbach, Ed) Academic Press, 1987, NY,
Methods in Enzymology: Immobilised Enzymes and Cells, Part B, Vol.137 (K. Mosbach, Ed) Academic Press, 1988, NY;
Immobilisation of enzymes and cells (GF Bickerstaff, Ed.) Humana Press, 1997, NJ, USA, Immobilised biomolecules in analysis: A practical approach (T Cass and FS Ligler, Eds) Oxford University Press, 1998, NY.
S. Nock, J.A Spudich and P. Wagner (1997) “Reversible, site-specific immobilisation of polyarginine-tagged fusion proteins on mica surfaces” FEBS Letters 141, 233-238)
B Jonsson, B Lindman, k. Holmberg and B Kronberg, Interaction of Polymers with Surfaces: in surfactant and polymers in aqueous solution, John Wiley and Sons, 1998, NY. G.P. Royer (1974) Supports for immobilized enzymes, CHEMTECH 4, 699
Y Harada, K Yasuda, S Nomura, n Kajimura and Y C Sasaki (1998) Vectorially Oriented Fixation of MembraneEmbedded Bacteriorhodopsin onto an Inert Base” Langmuir 14, 1829-1835.
Inouye, S et al. “Aequorea green fluorescent protein: Expression of the gene and fluorescence characteristics of the recombinant protein” FEBS Letters 341 (1994) pages 227-280.
Dyke, Michael W. Van; Sirito, Maro and Sawadogo, Michele “Single-step purification of bacterially expressed polypeptides containing an oligo-histidine domain”, Gene, Col.1 11, Issue 1,1 Feb.1992, pages 99-104.
Green fluorescent protein: properties, applications and protocols (m Chalfie, S kain, Eds.) Wiley-Liss, 1998, NY;
Protein localisation by fluorescence microscopy: a practical approach (VJ Allan, Ed.) Oxford University Press, 2000, NY; Imprint oxford; New York: Oxford University Press, c2000; Oancea, Diacylglycerol Signalling in Living Cells “The Journal of Cell Biology, Volume 140, No.3,
E et al “Green Fluorescent Protein (GFPytagged Cysteine-rich Domains from protein Kinase Cas Fluorescent Indications for February 9, 1998, pages 485-498.
J Sambrook and D W Russel, Molecular cloning: a laboratory manual Vol 1-3, Cold Spring Harbor Laboratory Press, 2001, Cold Spring Harbor.

### PROTOCOL I

### Plasmid or Cosmid DNA Purification Using QIAGEN® Plasmid Midi and Maxi Kits

Things to do Before Starting

Add the provided RNase A solution to Buffer P1 before use. Use one vial of RNase A (spin down briefly before use) per bottle of Buffer P1, to give a final concentration of 100 μg/ml.
Check Buffer P2 for SDS precipitation due to low storage temperatures.
If necessary, dissolve the SDS by warming to 37° C.
Pre-chill Buffer P3 to 4° C.
   Procedure

1. Pick a single colony from a freshly streaked selective plate and inoculate a starter culture of 2-5 ml LB medium containing the appropriate selective antibiotic. Incubate for 8 h at 37° C. with vigorous shaking (˜300 rpm).

2. Dilute the starter culture 1/500 to 1/1000 into selective LB medium. For high-copy plasmids inoculate 25 ml medium. For low-copy plasmids, inoculate 100 ml medium. Grow at 37° C. for 12-16 h with vigorous shaking (˜300 rpm).

3. Harvest the bacterial cells by centrifugation at 6000×g for 15 min at 4° C.

4. Resuspend the bacterial pellet in 4 ml of Buffer P1.

5. Add 4 ml of Buffer P2, mix gently but thoroughly by inverting 46 times, and incubate at room temperature for 5 min. Do not vortex, as this will result in shearing of genomic DNA. The lysate should appear viscous. Do not allow the lysis reaction to proceed for more than 5 min. After use, the bottle containing Buffer P2 should be closed immediately to avoid acidification from C0₂ in the air.

6. Add 4 ml of chilled Buffer P3, mix immediately but gently by inverting 4-6times, and incubate on ice for 15 min or 20 min.

7. Centrifuge at 20,000 ×g for 30 min at 4° C. Remove supernatant containing plasmid DNA promptly. After centrifugation the supernatant should be clear.

8. Centrifuge the supernatant again at 20,000 ×g for 15 min at 4° C. Remove supernatant containing plasmid DNA promptly.

9. Equilibrate a QIAGEN-tip 100 by applying 4 ml Buffer QBT, and allow the column to empty by gravity flow. Allow the QIAGEN-tip to drain completely.

10. Apply the supematant from step 8 to the QIAGEN-tip and allow it to enter the resin by aravitv flow.

11. Wash the QIAGEN-tip with 2×10 ml Buffer QC. Allow Buffer QC to move through the QIAGEN-tip by gravity flow.

12. Elute DNA with 5 ml Buffer QF. Collect the eluate in a 10 ml or 30 ml tube. Use of polycarbonate centrifuge tubes is not recommended as polycarbonate is not resistant to the alcohol used in subsequent steps. If you wish to stop the protocol and continue later, store the eluate at 4° C. Storage periods longer than ovemight are not recommended.

13. Precipitate DNA by adding 3.5 ml room-temperature isopropanol to the eluted DNA. Mix and centrifuge immediately at 15,000 ×g for 30 min at 4° C. Carefully decant the supematant. All solutions should be at room temperature in order to minimize salt precipitation, although centrifugation is carried out at 4° C to prevent overheating of the sample. Isopropanol pellets have a glassy appearance and may be more difficult to see than the fluffy, salt-containing pellets that result from ethanol precipitation. Marking the outside of the tube before centrifugation allows the pellet to be more easily located. Isopropanol pellets are also more loosely attached to the side of the tube, and care should be taken when removing the supernatant.

14. Wash DNA pellet with 2 ml of room-temperature 70% ethanol, and centrifuge at 15,000 ×g for 10 min. Carefully decant the supernatant without disturbing the pellet. The 70% ethanol removes precipitated salt and replaces isopropanol with the more volatile ethanol, making the DNA easier to redissolve.

15. Air-dry the pellet for 510 min, and redissolve the DNA in a suitable volume of buffer (e.g., TE buffer, pH 8.0, or 10 mM Tris-Cl, pH 8.5). Redissolve the DNA pellet by rinsing the walls to recover all the DNA, especially if glass tubes have been used. Pipetting the DNA up and down to promote resuspension may cause shearing and should be avoided. Overdrying the pellet will make the DNA difficult to redissolve. DNA dissolves best under slightly alkaline conditions; it does not easily dissolve in acidic buffers.

Determination of yield: To determine the yield, DNA concentration should be determined by both UV spectrophotometry and quantitative analysis on an agarose gel.

### PROTOCOL 2

### QlAquick Gel Extraction Kit Protocol using a Microcentrifuge

This protocol is designed to extract and purify DNA of 70 bp to 10 kb from standard or low-melt agarose gels in TAE or TBE buffer. Up to 400 mg agarose can be processed per spin column.

Notes: The yellow color of Buffer QG indicates a pH -7.5.
Add ethanol (96-100%) to Buffer PE before use (see bottle label for volume).
Isopropanol (100%) and a heating block or water bath at 500C are required.
All centrifugation steps are carried out at 13,000 rpm (˜17,900 ×g) in a conventional table-top microcentrifuge.
3 M sodium acetate, pH 5.0, may be necessary.

1. Excise the DNA fragment from the agarose gel with a clean, sharp scalpel. Minimize the size of the gel slice by removing extra agarose.

2. Weigh the gel slice in a colorless tube. Add 3 volumes of Buffer QG to 1 volume of gel (100 mg-100 μl). The maximum amount of gel slice per QlAquick column is 400 mg; for gel slices >400 mg use more than one QlAquick column.

3. Incubate at 50° C. for 10 min (or until the gel slice has completely dissolved). To help dissolve gel, mix by vortexing the tube every 2-3 min during the incubation. IMPORTANT: Solubilize agarose completely.

4. After the gel slice has dissolved completely, check that the color of the mixture is yellow (similar to Buffer QG without dissolved agarose). If the color of the mixture is orange or violet, add 10 pl of 3 M sodium acetate, pH 5.0, and mix. The color of the mixture will turn to yellow. The adsorption of DNA to the QlAquick membrane is efficient only at pH ˜7.5. Buffer QG contains a pH indicator which is yellow at pH ˜7.5 and orange or violet at higher pH, allowing easy determination of the optimal pH for DNA binding.

5. Add 1 gel volume of isopropanol to the sample and mix. This step increases the yield of DNA fragments <500 bp and >4 kb. For DNA fragments between 500 bp and 4 kb, addition of isopropanol has no effect on yield. Do not centrifuge the sample at this stage.

6. Place a QlAquick spin column in a provided 2 ml collection tube.

7. To bind DNA, apply the sample to the QlAquick column, and centrifuge for 1 min. The maximum volume of the column reservoir is 800 μl. For sample volumes of more than 800 pl, simply load and spin again.

8. Discard flow-through and place QlAquick column back in the same collection tube. Collection tubes are re-used to reduce plastic waste.

9. (Optional): Add 0.5 ml of Buffer QG to QlAquick column and centrifuge for 1 min. This step will remove all traces of agarose. It is only required when the DNA will subsequently be used for direct sequencing, in vitro transcription or microinjection.

10. To wash, add 0.75 ml of Buffer PE to QlAquick column and centrifuge for 1 min. Note: If the DNA will be used for salt sensitive applications, such as blunt-end ligation and direct sequencing, let the column stand 2-5 min after addition of Buffer PE, before centrifuging.

11. Discard the flow-through and centrifuge the QlAquick column for an additional 1 min at 13,000 rpm (˜17,900 ×g). IMPORTANT: Residual ethanol from Buffer PE will not be completely removed unless the flow-through is discarded before this additional centrifugation.

12. Place QlAquick column into a clean 1.5 ml microcentrifuge tube.

13. To elute DNA, add 50 μl of Buffer EB (10 mM Tris-Cl, pH 8.5) or H₂O to the center of the QlAquick membrane and centrifuge the column for 1 min. Alternatively, for increased DNA concentration, add 30 μl elution buffer to the center of the QlAquick membrane, let the column stand for 1 min, and then centrifuge for 1 min.

IMPORTANT: Ensure that the elution buffer is dispensed directly onto the QlAquick membrane for complete elution of bound DNA. The average eluate volume is 48 pI from 50 μl elution buffer volume, and 28 μl from 30 μl.

Elution efficiency is dependent on pH. The maximum elution efficiency is achieved between pH 7.0 and 8.5. When using water, make sure that the pH value is within this range, and store DNA at −20° C. as DNA may degrade in the absence of a buffering agent. The purified DNA can also be eluted in TE (10 mM Tris-Cl, 1 mM EDTA, pH 8.0), but the EDTA may inhibit subsequent enzymatic reactions.

### PROTOCOL 3

### QlAprep Spin Miniprep Kit Protocol using a MicrocentrifuAe

This protocol is designed for purification of up to 20 pg of high-copy plasmid DNA from 1-5 ml overnight cultures of E. coli in LB (Luria-Bertani) medium.

All protocol steps should be carried out at room temperature.

Procedure

1. Resuspend pelleted bacterial cells in 250 μl Buffer P1 and transfer to a microcentrifuge tube. Ensure that RNase A has been added to Buffer P1. No cell clumps should be visible after resuspension of the pellet.

2. Add 250 μl Buffer P2 and gently invert the tube 416 times to mix. Do not vortex, as this will result in shearing of genomic DNA. Do not allow the lysis reaction to proceed for more than 5 min.

3. Add 350 μl Buffer N3 and invert the tube immediately but gently 46 times. To avoid localized precipitation, mix the solution gently but thoroughly, immediately after addition of Buffer N3. The solution should become cloudy.

4. Centrifuge for 10 min at maximum speed in a tabletop microcentrifuge.

5. Apply the supernatants from step 4 to the QlAprep column by decanting or pipetting.

6. Centrifuge for 30-60 s. Discard the flow-through.

7. (Optional): Wash the QlAprep spin column by adding 0.5 ml Buffer PB and centrifuging for 30-60 s. Discard the flow-through. This step is necessary to remove trace nuclease activity when using endA+strains such as the JM series, HB101 and its derivatives, or any wild-type strain, which have high levels of nuclease activity or high carbohydrate content.

8. Wash QlAprep spin column by adding 0.75 ml Buffer PE and centrifuging for 30-60 seconds.

9.Discard the flow-through, and centrifuge for an additional 1 min to remove residual wash buffer. IMPORTANT: Residual wash buffer will not be completely removed unless the flow-through is discarded before this additional centrifugation. Residual ethanol from Buffer PE may inhibit subsequent enzymatic reactions.

10. Place the QlAprep column in a clean 1.5 ml microcentrifuge tube. To elute DNA, add 50 μl Buffer EB (10 mM Tris-Cl, pH 8.5) or water to the center of each QlAprep column, let stand for 1 min, and centrifuge for I min.

### PROTOCOL 4

### ProBond Purification System

### A Manual of Methods for Purification of Polyhistidine-containing Recombinant Proteins

ProBond resin should be stored at +4° C. All other components of the protocol may be stored at room temperature.

Additional Materials: All native purification buffers are prepared from the 5× Native Purification Buffer (250 mM Na₃PO₄; 2.5 M NaCI) and the 3 M Imidazole.

Resin and Column Specifications: ProBond resin is precharged with Ni²⁺ ions and appears blue in color. It is provided as a 50% slurry in 20% ethanol. Binding capacity of ProBond resin: 1.5 mg of protein per ml of resin Recommended flow rate: 0.5 ml/min. pH stability (long term): 3.13. pH stability (short term): 2.14

Binding Characteristics: ProBond, resin uses the chelating ligand iminodiacetic acid (IDA) in a highly cross-linked agarose matrix. IDA binds Ni2+ ions by three coordination sites.

Preparation of Native Purification Buffers

You must dilute and adjust the pH of the 5× Native Purification Buffer to create 1X Native Purification Buffer (50 mM Na₃PO₄; 500 mM NaCI; pH 8.0). From this, you can create the following buffers: Native Binding Buffer, Native Wash Buffer, Native Elution Buffer.

To prepare the native buffers, you will need the following reagents: 5× Native Purification Buffer, 3M Imidazole, NaOH, HCI, Sterile distilled water.

Imidazole Concentration in Native Buffers: Imidazole is included in the native wash and elution buffers to minimize the binding of untagged, contaminating proteins and increase the purity of the target protein with fewer wash steps. Note that, if your level of contaminating proteins is high, imidazole can also be added to the native binding buffer. If your protein does not bind well under these conditions, you can experiment with lowering or eliminating the concentration of imidazole in the buffers and increasing the number of wash and elution steps.

1× Native Purification Buffer: For the following recipes, you must dilute and adjust the pH of the 5× Native Purification Buffer (supplied in the kit) to create 1× Native Purification Buffer. To prepare 100 ml of 1× Native Purification Buffer, combine: 80 ml of H20, 20 ml of 5×=0 Native Purification Buffer. Adjust pH to 8.0 with NaOH or HCI

Native Binding Buffer Without Imidazole: Reserve 30 ml of the 1× Native Purification Buffer for use as the Native Binding Buffer (for column preparation, cell lysis, and binding).

Native Wash Buffer: To prepare 50 ml of Native Wash Buffer with 20 mM imidazole, combine; 50 ml of 1× Native Purification Buffer, 335 μl of 3M Imidazole. Adjust pH to 8.0 with NaOH or HCI.

Native Elution Buffer: To prepare 15 ml of Native Elution Buffer with 250 mM imidazole, combine: 13.75 ml of 1× Native Purification Buffer, 1.25 ml of 3M Imidazole. Adjust pH to 8.0 with NaOH or HCI.

Preparation of the ProBond Columns: Do not use strong reducing agents such as DTT with

ProBond columns. DTT reduces the nickel ions in the resin. In addition, do not use strong chelating agents such as EDTA or EGTA in the loading buffers or wash buffers, as these will strip the nickel from the columns. Be sure to check the pH of your buffers before starting

Column Preparation: When preparing a column as described below, make sure that the snap-off cap at the bottom of the column remains intact. To prepare a column:
1. Resuspend the ProBond, resin in its bottle by inverting and gently tapping the bottle repeatedly.
2. Pipet or pour 2 ml of the resin into a 1 0-ml Purification Column (Scale up the resin amount if necessary, considering the binding capacity of ProBond resin. All following steps are written for 2mL resin volume). Gently pellet it by low-speed centrifugation (1 minute at 800 ×g). Gently aspirate the supematant. Add 6 ml of sterile, distilled water and resuspend the resin by alternately inverting and gently tapping the column. Pipet or pour 2 ml of the resin into a 10-ml Purification Column. Gently pellet it by low-speed centrifugation (1 minute at 800 ×g). Gently aspirate the supernatant.
3. Add 6 ml of sterile, distilled water and resuspend the resin by alternately inverting and gently tapping the column.
4. Repellet the resin using centrifugation as described in Step 2, and gently aspirate the supernatant.
5. For purification under Native Conditions, add 6 ml of Native Binding Buffer (see the recipe on previous page).
6. Resuspend the resin by alternately inverting and gently tapping the column.
7. Repellet the resin using centrifugation as described in Step 2, and gently aspirate the supematant.

8. Repeat Steps 5 through 7.

Storing Prepared Columns: To store a column containing resin, add or 20% ethanol as a preservative and cap or paraflim the column. Store at room temperature.

Preparation of Bacteria Cell Lysate in Native Conditions

Follow the procedure below to prepare bacteria cell lysate under native conditions. Scale up or down as necessary.

1. Harvest cells from a 50 ml culture by centrifugation (e.g., 5000 rpm for 5 minutes in a Sorvall SS-34 rotor). Resuspend the cells in 8 ml of Native Binding Buffer.

2. Add 8 mg of lysozyme and incubate on ice for 30 minutes.

3. Using a sonicator equipped with a microtip, sonicate the solution on ice using six 10-second bursts at high intensity with a 10-second cooling period between each burst.

4. Centrifuge the lysate at 3,000 ×g for 15 min to pellet the cellular debris. Transfer the supematant to a fresh tube.

5. Store the lysate on ice or freeze at −20° C. Save 20 pl of the lysate at +40 C. for SDS-PAGE analysis

Introduction

In the following procedure, use the prepared Native Wash Buffer and Native Elution Buffer and the columns and cell lysate prepared under native conditions. Be sure to check the pH of your buffers before starting.

Purification Under Native Conditions

Using the native buffers, columns and cell lysate, follow the procedure below to purify proteins under native conditions (remember to scale up if necessary):

1. Add 8 ml of lysate to a prepared Purification Column.

2. Bind for 30-60 minutes using gentle agitation to keep the resin suspended in the lysate solution.

3. Settle the resin by low speed centrifugation (800 ×g), and carefully aspirate the supematant. Save supematant at +4° C. for SDS-PAGE analysis.

4. Wash with 8 ml Native Wash Buffer. Settle the resin by gravity or low speed centrifugation (800 ×g), and carefully aspirate the supematant. Save supernatant at +40 C. for SDS-PAGE analysis.

5. Repeat Step 4 three more times.

6. Clamp the column in a vertical position and snap off the cap on the lower end. Elute the protein with 8-12 ml Native Elution Buffer. Collect 0.5 or 1 ml fractions and analyze with SDS-PAGE.

6. Clamp the column in a vertical position and snap off the cap on the lower end. Elute the protein with 8-12 ml Native Elution Buffer. Collect 0.5 or I ml fractions and analyze with SDS-PAGE.

Note: Elution fractions should be stored at +40° C. If −200° C. storage is required, glycerol should be added to the fraction. For long term storage, protease inhibitors can be added.

Recharging ProBond. Resin

ProBond, resin can be used for up to three or four purifications of the same protein without recharging. We recommend not recharging the resin more than three times and only reusing it for purification of the same recombinant protein.

To recharge 2 ml of resin in a purification column:

1. Wash the column two times with 8 ml of 50 mM EDTA to strip away the chelated nickel ions.

2. Wash the column two times with 8 ml of 0.5M NaOH.

3. Wash the column two times with 8 ml of sterile, distilled water.

4. Recharge the column with two washes of 8 ml NiCI₂ hexahydrate at a concentration of 5 mg/ml prepared in sterile, distilled water.

5. Wash the column two times with 8 ml of distilled water.

6. Add 20% ethanol as a preservative and cap or parafllm the column. Store at room temperature.

1. Circular recombinant plasmid DNA construct encoding a) a protein tag, b) a visual marker protein, and containing c) a multiple cloning site suitable for insertion of an additional gene, characterised in that it further contains d) a frame adapter of variable length between the visual marker and protein tag genes and in that the gene sequence encoding the protein tag, the visual marker protein and the frame adaptor are specifically designed and engineered at the DNA level for respectively i) immobilisation purposes, ii) visualisation and quantification purposes at the protein level, and iii) providing a large distance separating protein and surface to enable the immobilised enzymes to display native-like characteristics.

2. Construct according to claim 1, characterised in that the protein tag is chosen from the group containing lysine (lys), histidine (his), tyrosine (tyr), phenylalanine (phe), arginine (arg), glutamic acid (glu), aspartic acid (asp), glutamate, aspartate, asparagine (asn), glycine (gly), glutamine (gin), alanine (ala), valine (val), tryptophan (trp).

3. Construct according to claim 1, characterised in that the protein tag is a histidine-tag such as a polyhistidine variant, in particular (6×) histidine.

4. Construct according to claim 1, characterised in that the visual marker protein is chosen from the group containing fluorescent or phosphorescent proteins, wherein the fluorescent protein is chosen from the group containing Green Fluorescent Protein (GFP), Red Fluorescent Protein (RFP), Yellow Fluorescent Protein (YFP) and Blue Fluorescent Protein (BFP) as well as their variants and/or mutants.

5. Construct according to claim 1, characterised in that the multiple cloning site contains restriction enzyme recognition sites chosen from the group containing Sacd, Sal I, Hid III, Eag I, Not I.

6. Construct according to claim 1, characterised in that it expresses a fusion protein, wherein the tag is suitable to interact directly with appropriate surface pendant groups of a support material.

7. Construct according to claim 6, characterised in that the direct interaction with the support material is covalent or non-covalent.

8. Construct according to claim 7, characterised in that the direct interaction is non-covalent and yet freely accessible and leach-free like covalent one.

9. Method for preparing and immobilising a protein on a support material, comprising:
a) Engineering at the DNA level, in series a protein tag suitable to interact directly with appropriate surface pendant groups of a support material, a fluorescent marker protein for visualisation and quantification purposes at the protein level and a multiple cloning site suitable for insertion of a target protein to be immobilised,
b) Inserting the corresponding gene of the target protein to be immobilised into the multiple cloning site;
c) Initiating protein expression.
d) Optionally pre-treating the support material;
e) Incubating the protein and support material together, wherein the protein is immobilised to the support via specific tag-surface interactions;
f) Washing away the non-specific biomolecules;
g) Optionally quantifying the fluorescence of the visual marker protein;
h) Optionally desorbing the target protein; characterised in that the support material is chosen from the group containing polymers, biopolymers, glass and composites containing silicone dioxides, metals and metal oxides, as well as any combination thereof on the microscopic, mesoscopic or macroscopic length scale.

10. Method according to claim 9, characterised in that the support material is chosen from the group containing polymers, silicon dioxides, aluminum oxides, titanium oxides, magnesium oxides, borates, metals and other metal oxides.

11. Method according to claim 9, characterised in that the polymers are chosen from the group containing polyolefins such as polystyrene, polyacrylates, polymethyacrylates, polybutylene, polyvinylalcohol and related derivatives, polyvinylchlorides, polyisoprene, polypropylene, polyphenols, polyamides, polyesters polysulfones, polyethersulfones, polyethersulfides, polyimines, polyethyleneglycols, polypropyleneglycols, polyimides, polycarbonates, polyurethanes.

12. Method according to claim 11, characterised in that the polymer surface is chemically treated to bear various functional groups chosen between carboxyl groups, hydroxyl groups, amino groups, amide groups, ester groups, imide groups, imine groups, mercapto groups, nitro groups, sulfonate groups, phosphate groups, phosphonate groups, cyano groups, sulfone groups, aldehyde groups, epoxide groups, urethane groups, ketone groups, phenolic groups, aromatic groups, alkyl, alkenyl, alkynyl, acyl and aryl groups, silanol groups, silicon oxide groups, siloxane groups, metal hydroxide groups, metal oxide groups, and elemental metals.

13. Method according to claim 9, characterised in that the support material is carboxylated polystyrene.

14. Method according to claim 9, characterised in that quantifying in step g) of the fluorescence of the visual marker protein is used in applications selected from analysis, diagnosis (like in enzyme based diagnostic kits), incubation, storage, sensing, arraying and orienting, catalysis, stabilisation, binding, signal transduction, chemical transformation, implant passivation and surface biocompatibilization, surface activation, purification, detoxification and scavenging.

15. A two-component system obtained by claim 9.

16. A two-component system according to claim 15, described by and an activated support material and a protein encoded by a recombinant plasmid DNA construct encoding a) a protein tag b) a visual marker protein and containing c) a multiple cloning site suitable for insertion of an additional gene, characterised in that it further contains d) a frame adapter of variable length between the visual marker and protein tag genes and in that the gene sequence encoding the protein tag, the visual marker protein and the frame adaptor are specifically designed and engineered at the DNA level for respectively i) immobilisation purposes. ii) visualisation and quantification purposes at the protein level, and iii) providing a large distance separating protein and surface to enable the imnmobilised enzymes to display native-like characteristics.


SEQUENCE LISTING (12 sequences)

SEQ ID NO: 1 - DNA, 733 bp, Aequorea victoria
  gene (17)..(733): GFP gene
ggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa

SEQ ID NO: 2 - DNA, 6029 bp, Artificial sequence
  Empty PETM-11 plasmid
  misc_feature (1)..(6029): Empty PETM-11 plasmid
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccggtaccactagttagagaccaagacacgccttgtgactgtcctgcagctttattctcttgatgctggtgctggaatagccctcatcactgccgaggctctgcatgctgccccgctcgtcagagtcgctcacactgctgctgctccagtccagatcacctgtgagatagtccgtgctctccacgtcaacgtcgatttcttccctgtcggagtcggagcgctccgaggagacggtggagccgatgctgtccatccggatcctctcaatgcccagcttctccagctgcctcttcaggtgtcgctgctctcgctgaagctggtcgatttggtgaacggcttttctgtcacaatcttcaagtttctttatgtgcaatttggcttttgttaataaactcaacgtagtgtgtcgacttgattcgggtcccagtggcaccagccccttcaacttctccaggcacaagcgaagatgagcccgtctattcttctccatttcattgtgagttgatctgctactgctgttattctttttggatttgttcctccgttttaaggcatctctgtccttgtttttgtatggtaacatggaggcataaccatgttcagcttctctctcccgccgctccagatagtcggccgcctccagcagcatctggatgttcatccgaaccgccgccgccatggcgccctgaaaataaagattctcagtagtggggatgtcgtaatcgctcatggggtgatggtgatggtgatgtttcatggtatatctccttcttaaagttaaatcaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - DNA, 5369 bp, Artificial sequence
  Intermediate pETM-adp plasmid, on way to pETM-GFP-Imm construct
catcaccatcaccatcaccccatgagcgattacgacatccccactactgagaatctttattttcagggcgccatgggaggcacggtaccggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgatttaactttaagaaggagatataccatgaaa

SEQ ID NO: 4 - DNA, 3337 bp, Artificial sequence
  pGFPuv plasmid coding for GFP from Aequorea victoria
  CDS (286)..(1014): pGFPuv plasmid coding for GFP from Aequorea victoria
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattccaactgagcgccggtcgctaccattaccaacttgtctggtgtcaaaaataataggcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 5 - protein, 239 aa, Artificial sequence
  Synthetic Construct
KMSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 6 - DNA, 6069 bp, Artificial sequence
  pETM-GFP-Imm plasmid containing Hisx6 tag, flexible joint as frame adapter, and A. victoria GFP gene
  CDS (1)..(876): pETM-GFP-Imm plasmid containing Hisx6 tag, flexible joint as frame adapter, and A. victoria GFP gene
atgaaacatcaccatcaccatcaccccatgagcgattacgacatccccactactgagaatctttattttcagggcgccatgggaggcacggtaccggtagaaaaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgatttaactttaagaaggagatatacc

SEQ ID NO: 7 - protein, 291 aa, Artificial sequence
  Synthetic Construct
MKHHHHHHPMSDYDIPTTENLYFQGAMGGTVPVEKMSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELRRQACGRTRAPPPPPLRSGC

SEQ ID NO: 8 - DNA, 17 bp, Artificial sequence
  Frame adapter used for prevention of frameshift mutation as a result of plasmid modification
  misc_feature (1)..(17): Frame adapter used for prevention of frameshift mutation as a result of plasmid modification
catgggaggcacggtac

SEQ ID NO: 9 - protein, 5 aa, Artificial sequence
  Peptide design based on size and flexibility to act as a linker between the tag and GFP protein segments
  MISC_FEATURE: Peptide design based on size and flexibility to act as a linker between the tag and GFP protein segments
MGGTV

SEQ ID NO: 10 - protein, 6 aa, Artificial sequence
  Peptide design based on charge and shape to bind the expressed protein to a suitably interactive surface
  MISC_FEATURE: Peptide design based on charge and shape to bind the expressed protein to a suitably interactive surface
HHHHHH

SEQ ID NO: 11 - protein, 238 aa, Aequorea victora
  MISC_FEATURE: Green fluorescent peptide coded by pGFPuv plasmid, permitting easy visualisation and quantification based on fluorescence properties
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 12 - protein, 291 aa, Artificial sequence; identical to SEQ ID NO: 7
  Completed peptide based on Hisx6 tag, short physically flexible linker and green fluorescent protein coded by pETM-GFP-Imm to impart fluorescence properties, allowing easy immobilisation with retention of bioactivity, visualisation and quantification
  MISC_FEATURE: Completed peptide based on Hisx6 tag, short physically flexible linker and green fluorescent protein coded by pETM-GFP-Imm to impart fluorescence properties, allowing easy immobilisation with retention of bioactivity, visualisation and quantification